(19) United States
(12) Patent Application Publication
(10) Pub. No.: US 2008/0301830 A1
(43) Pub. Date: Dec. 4, 2008

(54) Warted pumpkin

(76) Inventor: Roy Murdoch Pearman, Holland, MI (US)

Correspondence Address: King & Partners, PLC, 170 College Avenue, SUITE 230, HOLLAND, MI, 49423, US

(21) Appl. No.: 11/999,153

(22) Filed: Dec. 4, 2007

Related U.S. Application Data
(60) Provisional application No. 60/872,638, filed on Dec. 4, 2006.

(51) Int. Cl. A01H 5/00; A01H 1/02

(52) U.S. Cl. 800/260; 800/310

(57) ABSTRACT

A warted pumpkin having a body which includes an inner surface and an outer shell, wherein the outer shell includes at least one wart associated with the outer shell of the body.

### CROSS-REFERENCE TO RELATED APPLICATION(S)

This application claims the benefit of U.S. Provisional Application Ser. No. 60/872,638, filed Dec. 4, 2006, entitled “WARTED PUMPKIN,” which is hereby incorporated herein by reference in its entirety, including all references cited therein.

### BACKGROUND OF THE INVENTION

1. Field of the Invention

The present invention relates in general to pumpkins, and more particularly to pumpkins which include a plurality of warts on the outer surface of their shell.

2. Background Art

Pumpkins have been known in the art for years. Typically, in the United States, pumpkins are enjoyed throughout the fall holidays including, Halloween and Thanksgiving. For example, during the above-identified holidays many people place a plurality of pumpkins near the entrance of their house and/or decorate the inside of their house using pumpkins. Additionally, pumpkins are carved for Halloween—including the Jack-O-Lantern type.

While the commercial demand for pumpkins remains strong for the Halloween and Thanksgiving holidays, there remains an ever present desire to augment the present variety with new ornamental fruits (i.e. pumpkins) toward diversifying the market for consumers. One diversification type pumpkin that would appear to have great demand for Halloween is a warted pumpkin that embodies the weird, ugly and/or spooky spirit of Halloween as is celebrated by millions of Americans.

To the best of Applicant's knowledge, while blemished pumpkins do appear to occur naturally, they only occur at a very low frequency in occasional pumpkin populations and variety. In such occurrences, the blemishes on the shell of the pumpkin appear to be very small, smooth, and infrequent, such as is depicted in FIG. 1.

It is therefore an object of the present invention to provide a new warted pumpkin toward diversification of the same fruit family.

It is a further object of the present invention to provide a new pumpkin characterized by its warted shell surface and color of the same.

It is another object of the present invention to provide plants, tissue, and seeds for growing warted pumpkins.

It is still another object of the present invention to provide methods for growing warted pumpkins.

These and other objects of the present invention are disclosed herein per the specification, claims, and associated drawings.

### SUMMARY OF THE INVENTION

In one embodiment, the present invention is directed to a warted pumpkin, comprising: (a) a body having an inner surface and an outer shell, wherein the outer shell comprises a surface area; and (b) at least one wart associated with the outer shell of the body. It will be understood that nested warts may be present on the outer shell of the body.

In a preferred embodiment of the present invention, the at least one wart comprises at least approximately 5 percent of the surface area of the outer shell, and more preferably at least approximately 10 percent of the surface area of the outer shell, and yet more preferably at least approximately 25 percent of the surface area of the outer shell, and most preferably at least approximately 50 percent of the surface area of the outer shell.

In another preferred embodiment of the present invention, the at least one wart comprises a height, a width, and a depth, wherein the height and/or the width is greater in dimension than the depth.

In yet another preferred embodiment of the present invention, the at least one wart comprises a height, a width, and a depth, wherein the height and/or the width is greater in dimension than the depth by a ratio of approximately 2:1, and more preferably by a ratio of approximately 5:1.

In accordance with the present invention, the at least one wart may comprise a color that is substantially the same as the outer shell or not substantially the same as the outer shell.

The present invention is also directed to a warted pumpkin patch, comprising a field population, wherein at least approximately 10 percent of the field population comprises a warted pumpkin as is disclosed infra, and more preferably at least approximately 25 percent, and yet more preferably at least approximately 50 percent, and most preferably at least approximately 75 percent.

In a preferred embodiment of the present invention, the warted pumpkin comprises a Cucurbita pepo and/or maxima.

In another preferred embodiment of the present invention, the warted pumpkin comprises a Connecticut Field shape.

In yet another preferred embodiment of the present invention, the warted pumpkin does not comprise a Connecticut Field shape, and comprises a top that is not indented.

The present invention is further directed to: (a) a plant capable of producing a warted pumpkin as disclosed infra; (b) a seed capable of germinating into a plant capable of producing a warted pumpkin as disclosed infra; and/or (c) tissue from a plant producing a warted pumpkin as disclosed infra.

The present invention is also directed to a method for producing a new variety of plant, the method comprising the steps of: (a) pollinating one or more plant(s) of a variety capable of producing warted pumpkins as disclosed infra with another variety of Cucurbitacae plant to produce fruit with seeds; and (b) germinating the seeds to produce the new variety of plant.

### BRIEF DESCRIPTION OF THE DRAWINGS/PHOTOGRAPHS

It will be understood that the file of this patent contains at least one color photograph. Copies of this patent with color photographs will be provided by the Patent and Trademark Office upon request and payment of necessary fee.

Certain embodiments of the present invention are illustrated by the accompanying figures. It will be understood that the figures are not necessarily to scale and that details not necessary for an understanding of the invention or that render other details difficult to perceive may be omitted. It will be understood that the invention is not necessarily limited to the particular embodiments illustrated herein.

The invention will now be described with reference to the drawings and photographs wherein:

FIG. 1 of the drawings is a fragmented, cross-sectional representation of a conventional, non-warted pumpkin;

FIG. 2 of the drawings is a fragmented, cross-sectional representation of a warted pumpkin in accordance with the present invention; and

FIGS. 3-19 are color photographs of warted pumpkins prepared in accordance with the present invention.

### DETAILED DESCRIPTION OF THE INVENTION

While this invention is susceptible of embodiment in many different forms, there is shown in the drawings/photographs and will be described in detail, a specific embodiment with the understanding that the present disclosure is to be considered as an exemplification of the principles of the invention and is not intended to limit the invention to the embodiment illustrated.

It will be understood that like or analogous elements and/or components, referred to herein, may be identified throughout the drawings and/or photographs with like reference characters.

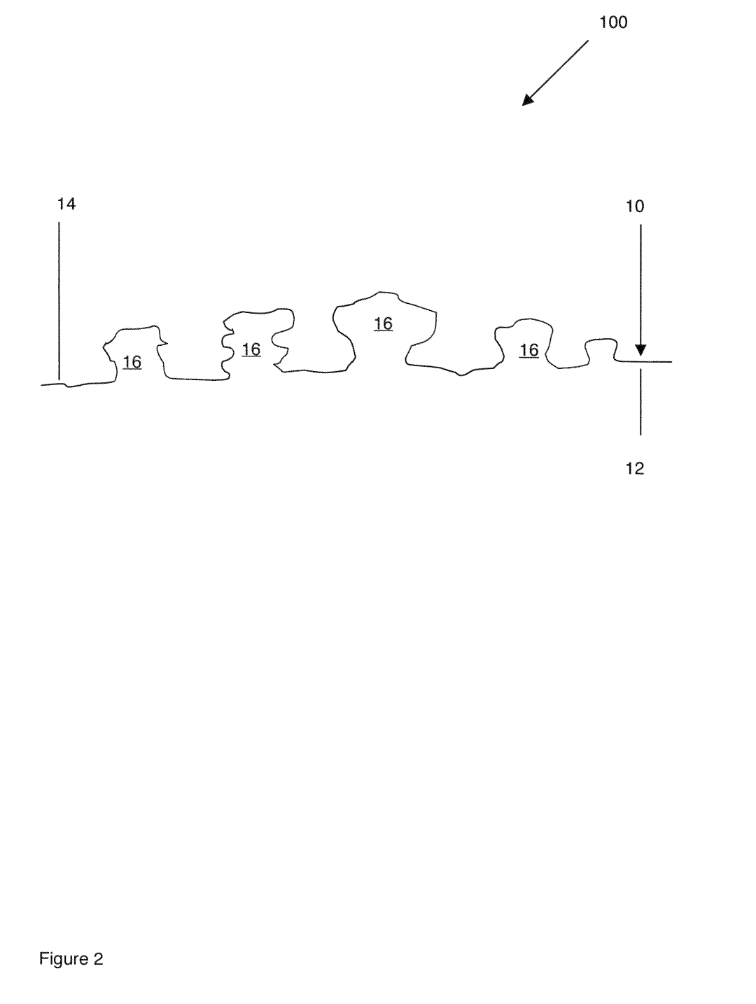

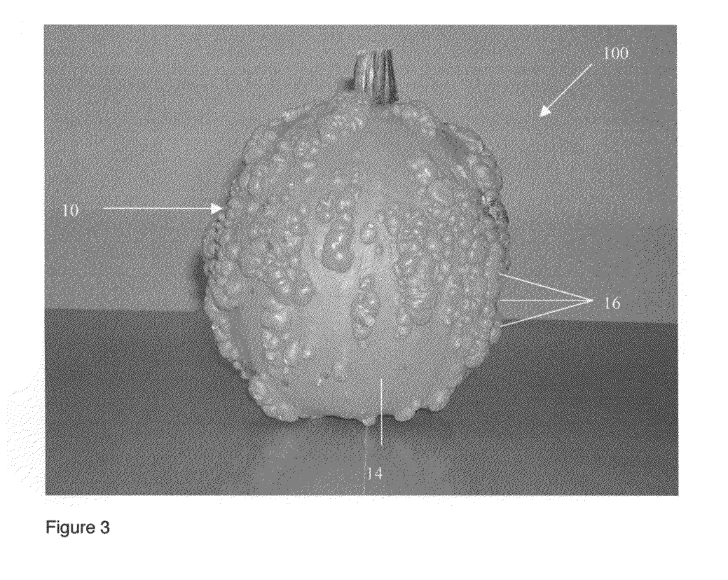

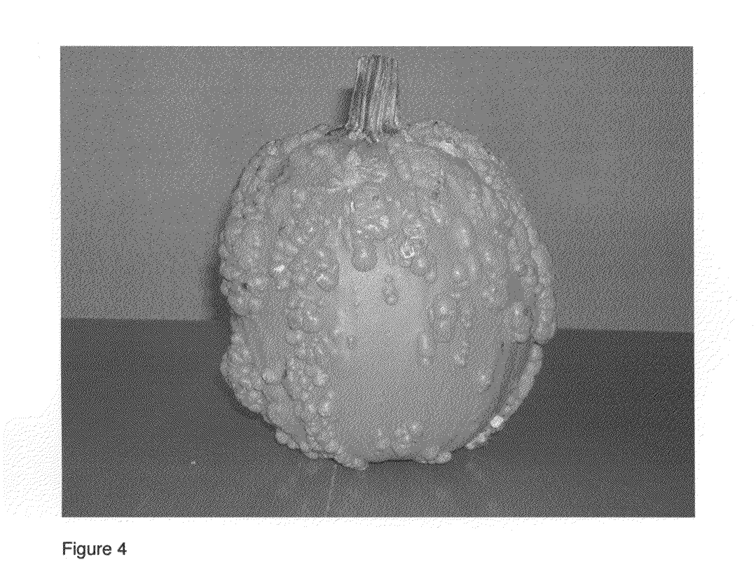

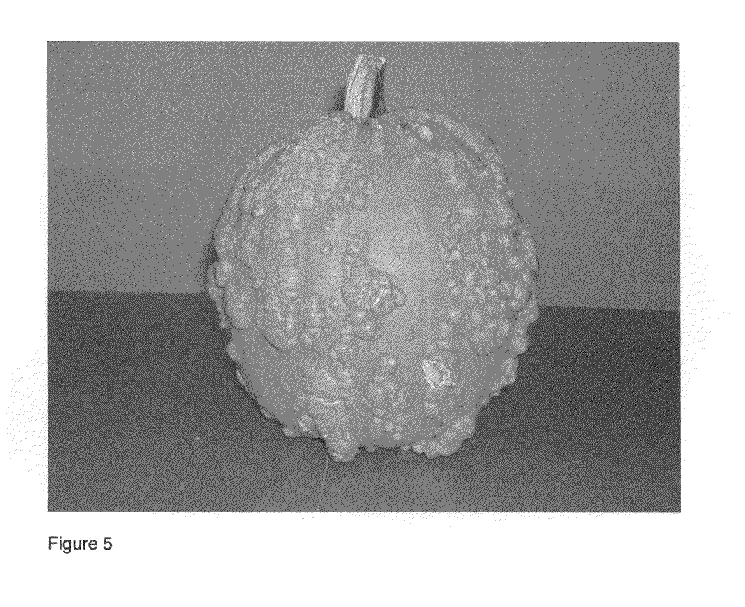

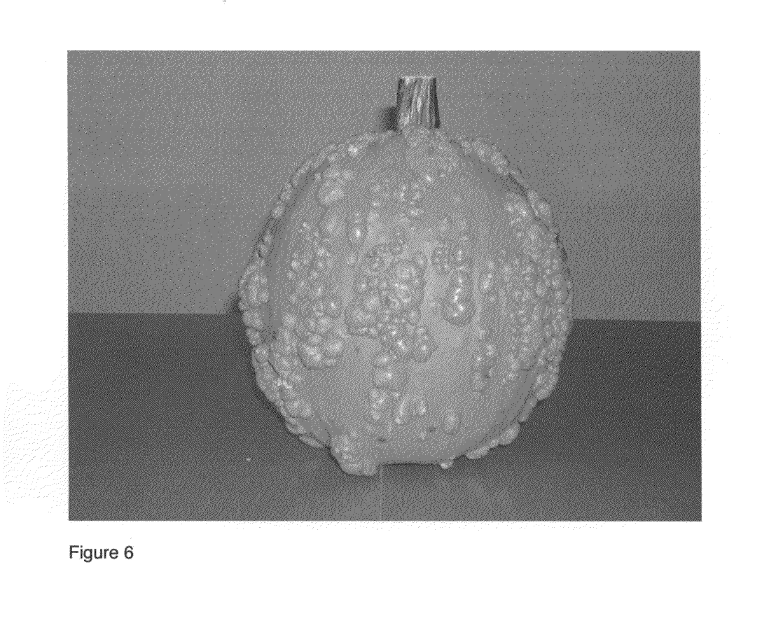

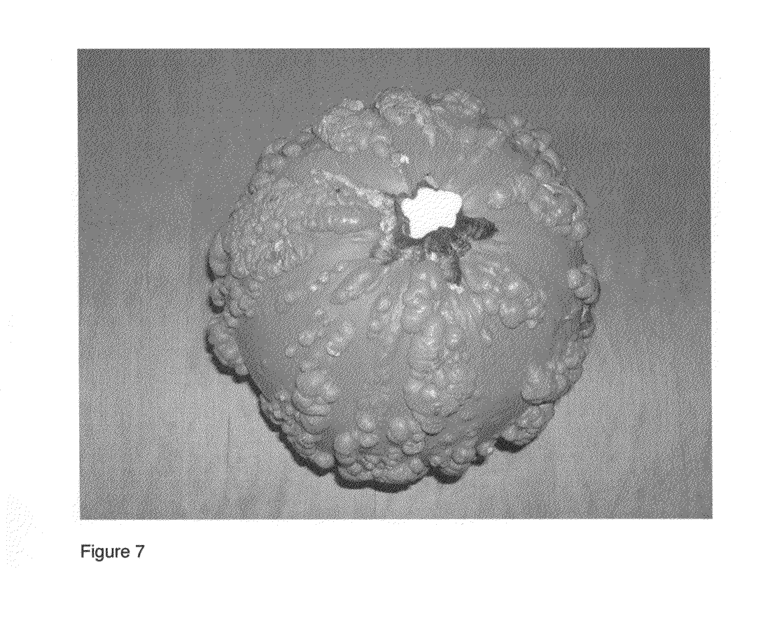

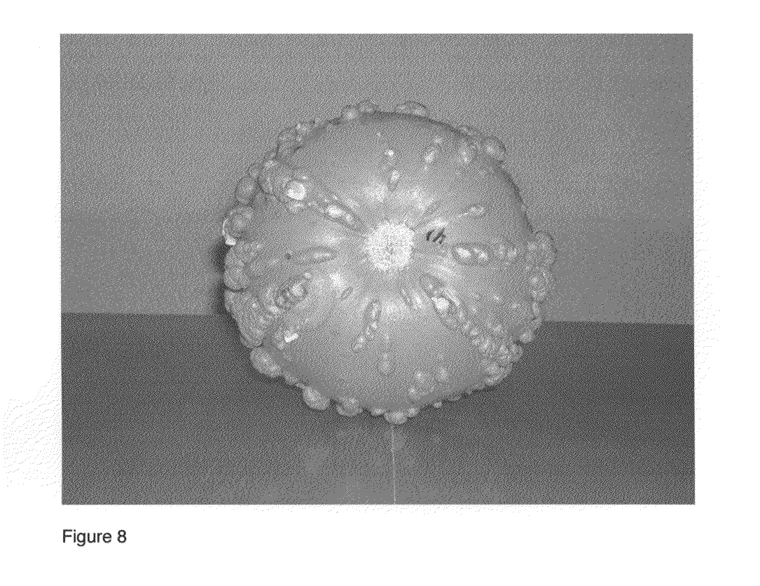

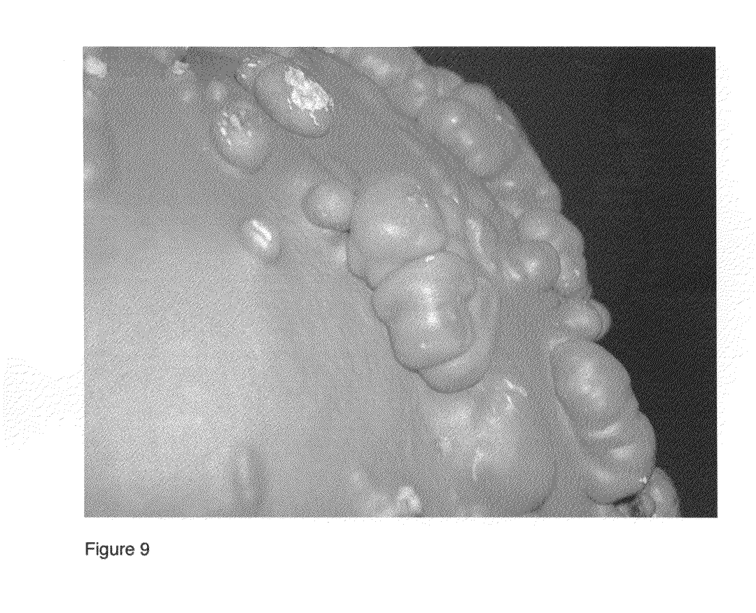

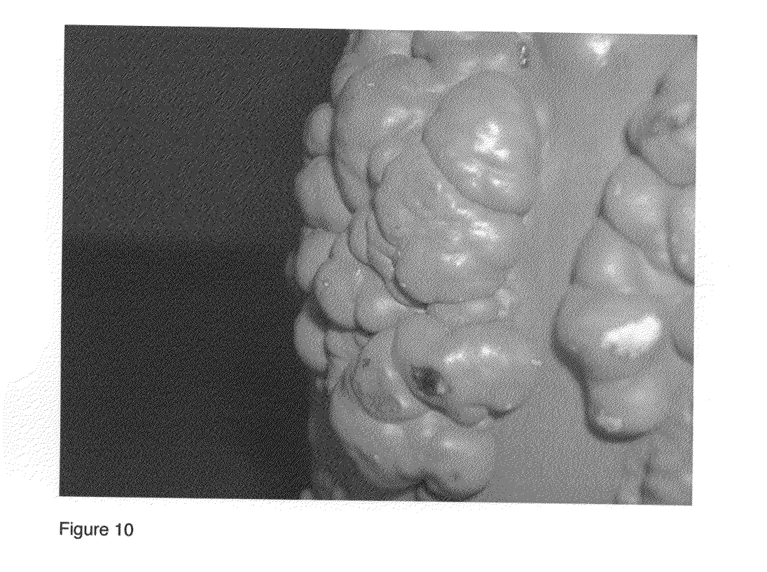

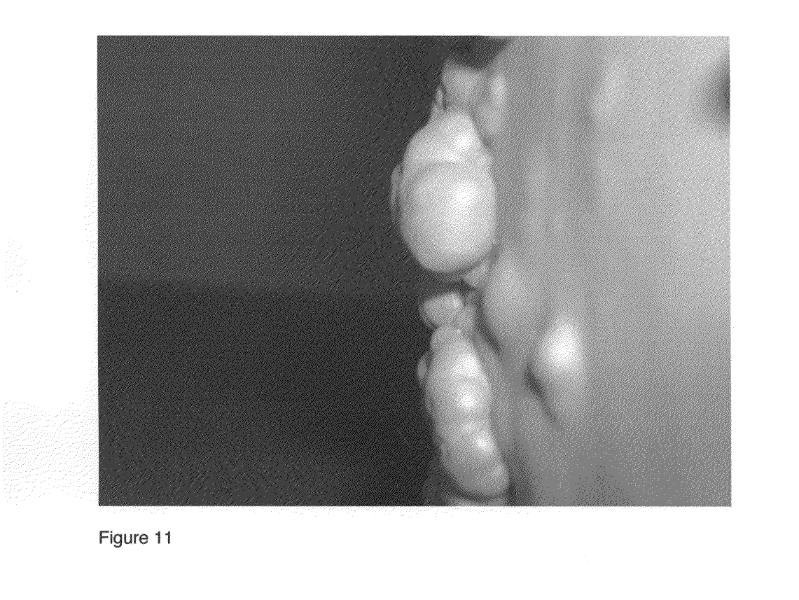

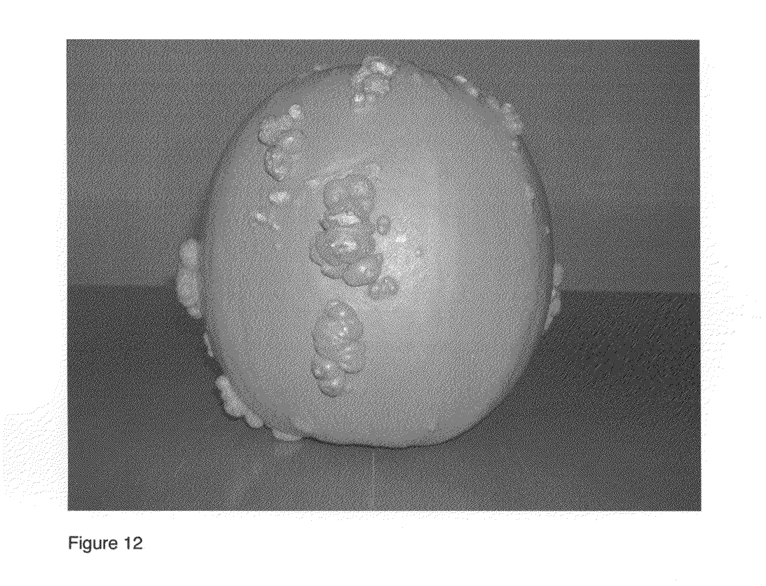

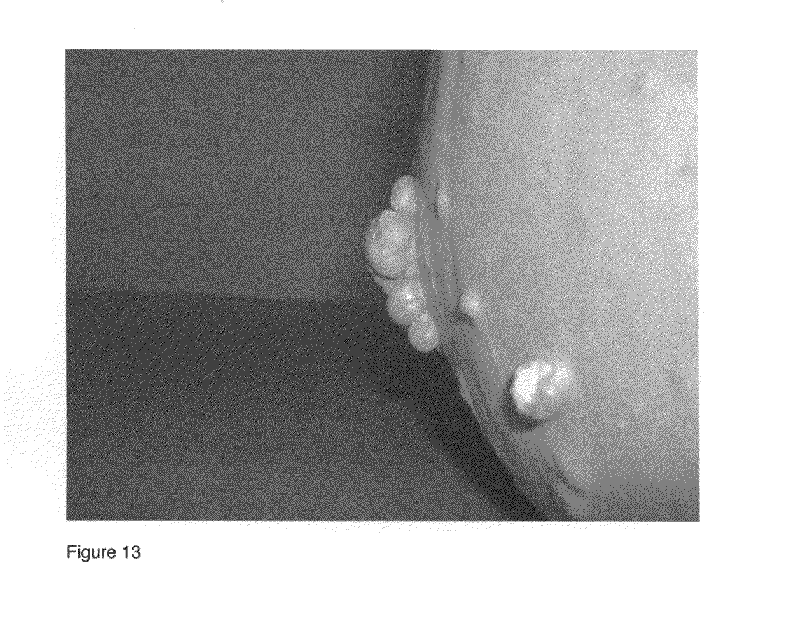

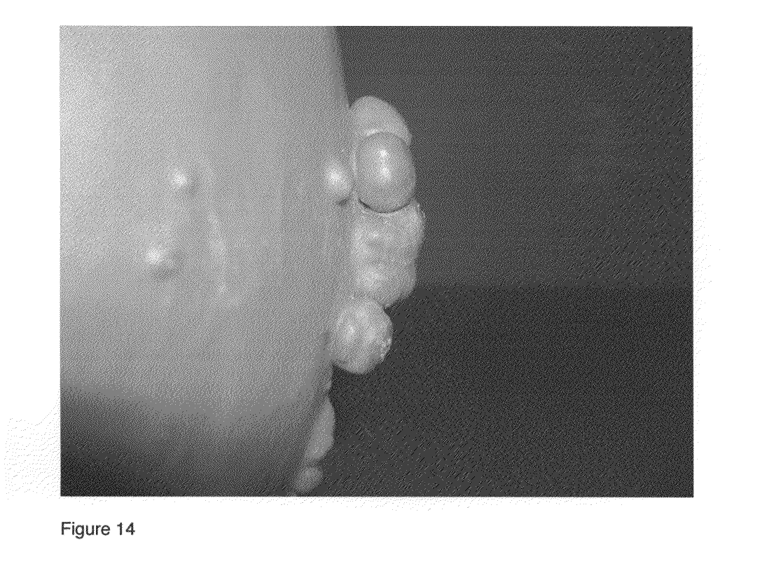

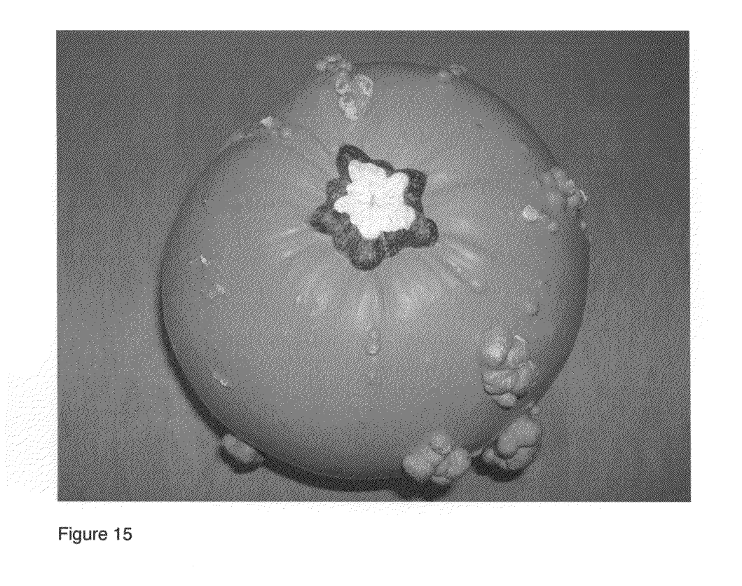

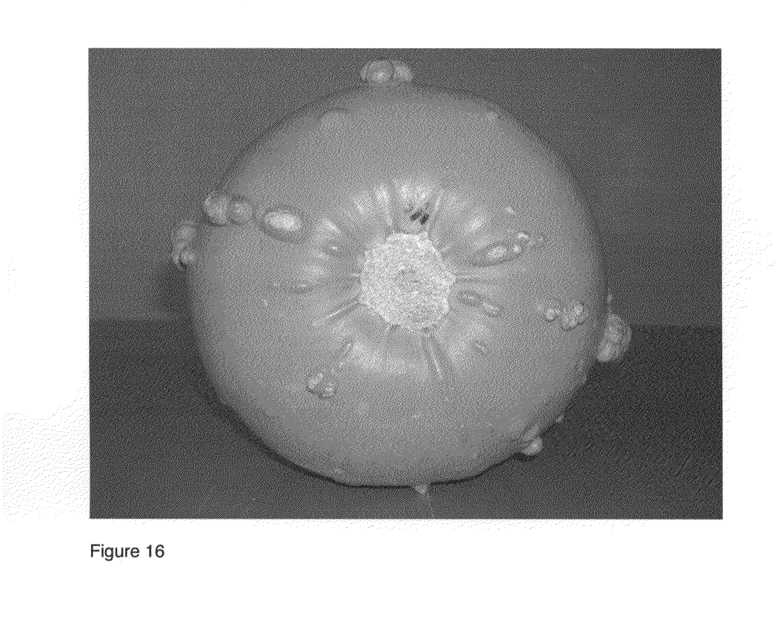

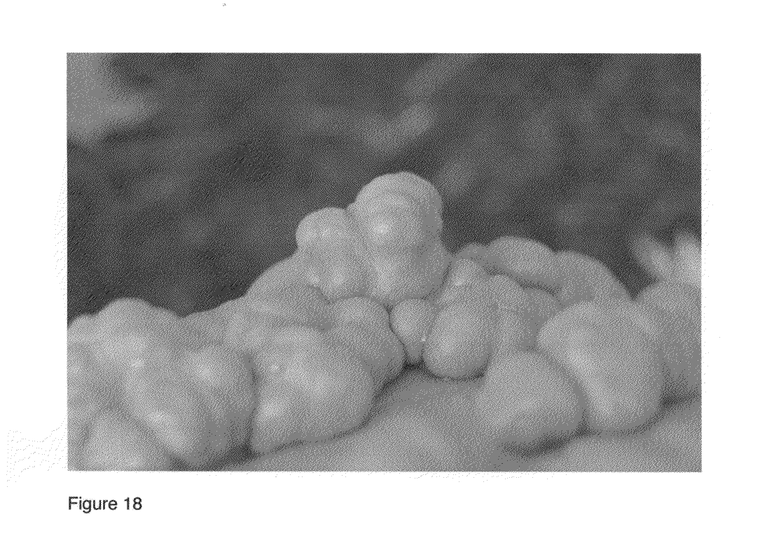

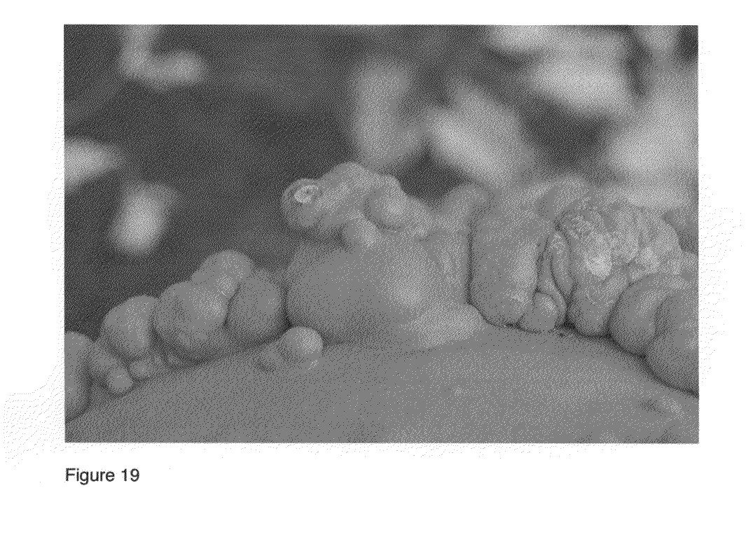

Referring now to the drawings and photographs and to FIGS. 2-19, collectively, the present invention is directed to warted pumpkin 100 which generally comprises body 10 having inner surface 12 and outer shell 14, wherein outer shell 14 comprises a surface area and one or more wart(s) 16 associated with outer shell 14 of body 10. It will be understood that nested warts may be present on the outer shell of the body as is best shown in FIGS. 18 and 19. Such nested warts may be double, triple, quadruple, and/or multiply nested—just to name a few.

Preferably warts 16 range in: (a) width from approximately 1 cm to approximately 10 cm; (b) height from approximately 0.5 cm to approximately 10 cm; and/or (c) depth (i.e. distance away from the outer shell) from approximately 0.5 cm to approximately 5 cm. It will be understood that while specific dimensions have been disclosed for illustrative purposes only, any one of a number of dimensions are likewise contemplated in accordance with the present invention.

In one embodiment of the present invention, it will be understood that warts 16 comprise at least approximately 5 percent of the surface area of the outer shell, more preferably at least approximately 10 percent of the surface area of the outer shell, yet more preferably at least approximately 25 percent of the surface area of the outer shell, and most preferably at least approximately 50 percent of the surface area of the outer shell.

In accordance with the present invention, each wart 16 comprises a height, a width, and a depth. Preferably, the height and/or the width of the wart is greater in dimension than the depth of the wart, and more preferably the height and/or the width of the wart is greater in dimension than the depth of the wart by a ratio of approximately 2:1, and most preferably by a ratio of approximately 5:1. It will be further understood that the height and/or width of the base of the wart attached to the shell of the pumpkin is preferably less than the height and/or width of the wart at a distance or depth from the shell (e.g. 0.5-5 cm). Such a configuration is best depicted in FIG. 2 and/or the associated photographs.

Warts 16 of warted pumpkin 100 may comprise any one of a number of colors including, orange, black, brown, green—just to name a few. Warted pumpkins of the present invention may also comprise a Cucurbita pepo and/or maxima and comprise a Connecticut Field shape. Alternatively, warted pumpkins of the present invention may not comprise a Connecticut Field shape, and include a top that is not indented.

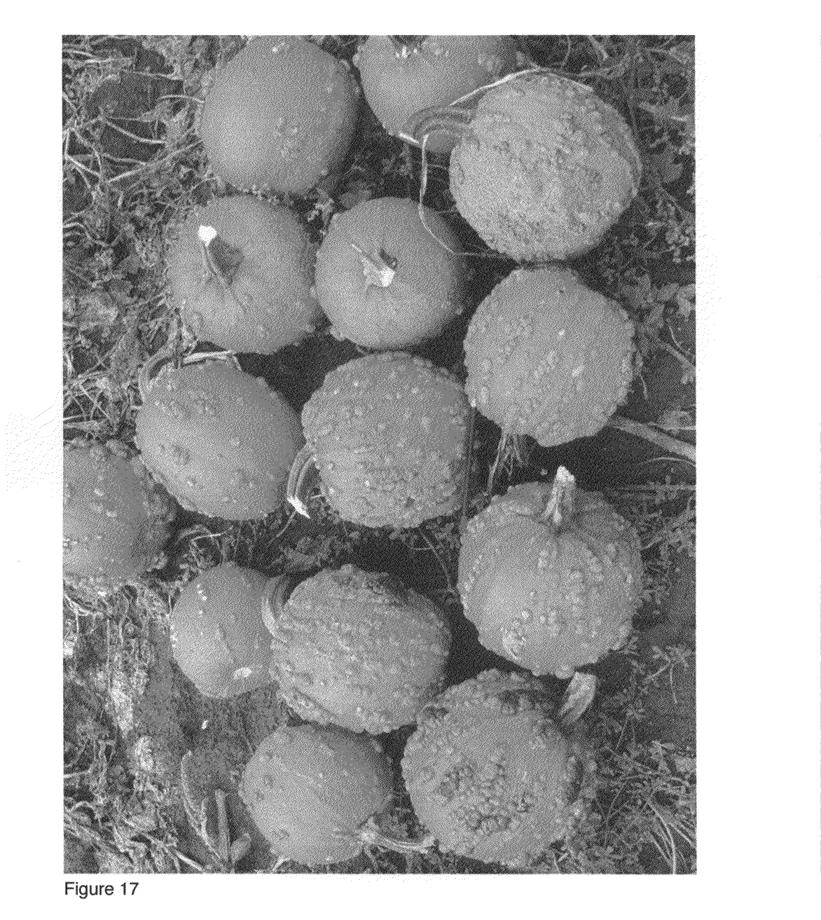

The present invention is also directed to a warted pumpkin patch (See FIG. 17), comprising a field population, wherein at least approximately 10 percent of the field population comprises a warted pumpkin as is disclosed herein. Preferred warted pumpkin field populations include, for example, greater than approximately 25 percent, greater than approximately 50 percent, and greater than approximately 75 percent—depending upon a plurality of factors including the location of the field population, local weather patterns, air and/or soil variations, just to name a few.

The present invention is further directed to a method for producing a new variety of plant, comprising the steps of: (a) pollinating a plant or plants of a variety capable of producing warted pumpkins with another variety of Cucurbitacae plant to produce fruit with seeds; and (b) germinating the seeds to produce the new variety of plant.

Provided herein below is one methodology for preparing warted pumpkins in accordance with the present invention. It will be understood that requisite materials are available from Siegers Seed Company in Holland, Mich.

### September 2002

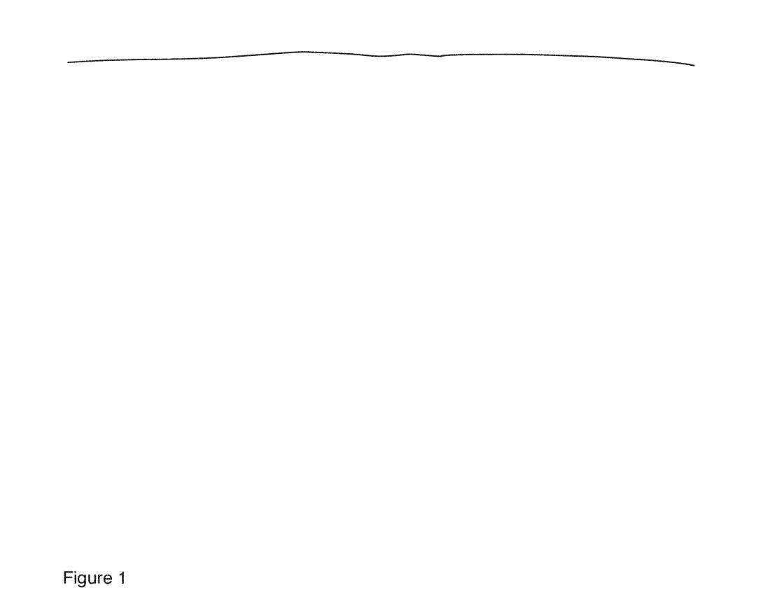

In a large commercial field of multiple unknown pumpkin varieties, a single fruit was discovered displaying a greater degree of warting than has ever been observed in prior experience by the inventor. On rare occasions in years prior to this discovery, pumpkin fruits had been observed to possess rumpled or bumpy surfaces as described in FIG. 1. The discovered fruit had a high frequency of bumpy skin as described in FIG. 1, and a lower percentage of warting as described in FIG. 2. The fruit was collected and seed was saved.

In the following timeline, the growing season for the respective hemisphere is described with northern hemisphere as (N) and southern hemisphere as (S). Northern hemisphere production and selection is carried out at Siegers Seed Company research farm in Holland, Mich. while Southern Hemisphere production and selection is carried out by Hybrid Seed Company at its research facility at Pukekohe, New Zealand. This dual hemisphere selection operation gives a wider selection for adaptability through multi-environment selection.

### 2003 N

Approximately 100 seeds from the mutant fruit were planted in isolation for observation. A vast degree of variability (segregation of genes) was noted, and 4 open-pollinated fruit were selected. The selections were solely based upon large size of fruit and/or significant warting.

Selection 78-01: round to slightly flattened shape, small size, 10 to 15 lbs.

Selection 78-02: round to slightly tall, medium size, 15 to 20 lbs.

Selection 78-03: elongated shape, large size, 20 lbs plus.

Selection 78-04: round shape, tight ribbing pattern, large size, 20 lbs plus.

### 2004 S

15 to 20 seeds from each of the 4 selections were sown and self-pollinations were made on every plant to begin an inbred line development program. Of the self-pollinations made, 11 fruits were selected based upon the warting characteristics that had developed on the fruits.

### 2004 N

Approximately 10 plants from each of the self-pollinations were grown. These were evaluated on Sep. 13, 2004, and the results were as follows:

| | Earli- | Powdery | | | | |
|---|---|---|---|---|---|---|
| Line | ness | Mildew | Habit | Texture | Comments | Selected |
| 78-01-1 | 1 | 1 | vine | small warts | none | yes |
| 78-01-2 | 5 | n/a | vine | smooth | none | no |
| 78-01-3 | 10 | 6 | s-bush | few warts | small fruit | yes |
| 78-02-1 | 2 | n/a | vine | smooth | good fruit | no |
| 78-02-2 | 3 | n/a | vine | smooth | small fruit | no |
| 78-03-1 | 2 | 1 | vine | heavy wart | poor fruit | yes |
| 78-03-2 | 6 | n/a | vine | smooth | round | no |
| 78-03-3 | 4 | n/a | vine | smooth | small | no |
| 78-04-1 | 7 | 5 | vine | bumpy | big fruit | yes |
| 78-04-2 | 7 | 10 | bush | few warts | still green | yes |
| 78-04-3 | n/a | 7 | vine | few warts | flat/yellow | yes |

1-10 scale
1 = very early maturing
10 = very susceptible to powdery mildew

### 2005 S

25 seed from 5 self-pollinations and 5 open-pollinated selections were sown and the resulting plants were both self and cross-pollinated. A total of 50 fruits were chosen at maturity for selection to the next stage for breeding. In addition to these controlled pollinations, two of the lines were cross-pollinated with HSC3359, a Hybrid Seed Company (HSC) Cucurbita pepo tester line, with good combining ability to evaluate heterosis and the inheritance of the warting characteristic.

### 2005 N

10 seeds from each of the 42 selections made in the Southern Hemisphere together with 8 open-pollinated fruits selected in 2004 Northern Hemisphere were sown with the 2 HSC test crosses. The resulting plants were self-pollinated to produce 1 to 2 fruits per line. From the hybrid assessment it was observed that both hybrids expressed the warting characteristic but not all other desired characteristics.

### 2006 S

15 to 20 seeds from each of the 14 selections from 2005 southern hemisphere fruits along with 13 selections from 2005 northern hemisphere fruits were sown. 50 self-pollinated fruits were produced after further selection from these. Additionally, 12 crosses were made between these warted lines and introduced material from the HSC Cucurbita pepo germplasm collection to develop further breeding populations. The aim of these crosses was to widen the genetic base and to improve the characteristics of fruit size, disease tolerance, stem quality, skin color and appearance. From the most advanced lines with improved uniformity, crosses were again made to develop a new set of hybrids using new testers with high general combining ability.

### 2006 N

32 breeding lines were sown to continue inbred line development and further selection. 16 HSC hybrids and controls were also sown for evaluation. From the resulting self-pollination of the breeding lines, 22 self-pollinated fruits were selected. From the hybrids evaluated, 2 were deemed to be of value for future release.

### 2007 S

35 breeding lines and populations were sown (15 to 20 seeds each) to continue inbred line development and selection. Selections from 51 self-pollinations were made together with 12 new HSC hybrids. Trial quantities were produced of the two selected hybrids using more highly developed inbred material for advanced trials.

### 2007 N

39 breeding lines and 12 hybrids were sown resulting in 45 self-pollinated fruits being selected. Observation and evaluation of the advanced test hybrids was conducted. Additional hybrids were identified for trials in 2008. Multiple trials over the Midwest, Northeast, and Mid-Atlantic territory of the US of the current invention showed the two hybrids to be stabilized and ready for release. The two hybrids will be put into seed production in the Southern hemisphere. Names for the two hybrids have been registered with the USDA: “Goose Bumps” for the Pie-sized hybrid and “Knuckle Head” for the mid-sized variety.

With regard to growing the pumpkins of the present invention, reference is also made to U.S. Pat. No. 6,300,546, including all the references cited therein, which is hereby incorporated herein by reference in its entirety.

The foregoing description merely explains and illustrates the invention and the invention is not limited thereto except insofar as the appended claims are so limited, as those skilled in the art who have the disclosure before them will be able to make modifications without departing from the scope of the invention.

What is claimed is:

1. A warted pumpkin, comprising:
a body having an inner surface and an outer shell, wherein the outer shell comprises a surface area; and
at least one wart associated with the outer shell of the body.

2. The warted pumpkin according to claim 1, wherein the at least one wart comprises at least approximately 5 percent of the surface area of the outer shell.

3. The warted pumpkin according to claim 1, wherein the at least one wart comprises at least approximately 10 percent of the surface area of the outer shell.

4. The warted pumpkin according to claim 1, wherein the at least one wart comprises at least approximately 25 percent of the surface area of the outer shell.

5. The warted pumpkin according to claim 1, wherein the at least one wart comprises at least approximately 50 percent of the surface area of the outer shell.

6. The warted pumpkin according to claim 1, wherein the at least one wart comprises a height, a width, and a depth, and further wherein at least one of the height and the width is greater in dimension than the depth.

7. The warted pumpkin according to claim 1, wherein the at least one wart comprises a height, a width, and a depth, and further wherein the height and the width are greater in dimension than the depth.

8. The warted pumpkin according to claim 1, wherein the at least one wart comprises a height, a width, and a depth, and further wherein at least one of the height and the width is greater in dimension than the depth by a ratio of approximately 2:1

9. The warted pumpkin according to claim 1, wherein the at least one wart comprises a height, a width, and a depth, and further wherein the height and the width are greater in dimension than the depth by a ratio of approximately 2:1.

10. The warted pumpkin according to claim 1, wherein the at least one wart comprises a height, a width, and a depth, and further wherein at least one of the height and the width is greater in dimension than the depth by a ratio of approximately 5:1

11. The warted pumpkin according to claim 1, wherein the at least one wart comprises a height, a width, and a depth, and further wherein the height and the width are greater in dimension than the depth of the wart by a ratio of approximately 5:1.

12. The warted pumpkin according to claim 1, wherein the at least one wart comprises at least two warts nested on top of one another.

13. The warted pumpkin according to claim 1, wherein the at least one wart comprises a color that is substantially the same as the outer shell.

14. The warted pumpkin according to claim 1, wherein the at least one wart comprises a color that is not substantially the same as the outer shell.

15. A warted pumpkin patch, comprising:
a field population, wherein at least approximately 10 percent of the field population comprises a warted pumpkin according to claim 1.

16. A warted pumpkin patch, comprising:
a field population, wherein at least approximately 25 percent of the field population comprises a warted pumpkin according to claim 1.

17. A warted pumpkin patch, comprising:
a field population, wherein at least approximately 50 percent of the field population comprises a warted pumpkin according to claim 1.

18. A warted pumpkin patch, comprising:
a field population, wherein at least approximately 75 percent of the field population comprises a warted pumpkin according to claim 1.

19. The pumpkin of claim 1, wherein the pumpkin comprises Cucurbita pepo.

20. The pumpkin of claim 1, wherein the pumpkin has a Connecticut Field shape.

21. The pumpkin of claim 1, wherein the pumpkin is not a Connecticut Field shape, and has a top that is not indented.

22. A plant capable of producing a warted pumpkin according to claim 1.

23. A seed capable of germinating into a plant capable of producing a warted pumpkin according to claim 1.

24. A tissue from a plant producing a warted pumpkin according to claim 1.

25. A method for producing a new variety of plant, the method comprising the steps of:
pollinating at least one plant of a variety capable of producing warted pumpkins with another variety of Cucurbitacae plant to produce fruit with seeds; and
germinating the seeds to produce the new variety of plant.
